(19) United States
(12) Patent Application Publication
(10) Pub. No.: US 2006/0217757 A1
(43) Pub. Date: Sep. 28, 2006

(54) Device and method of weight control via indirect abdominal cavity volume reduction

(76) Inventor: Gary M. Horndeski, League City, TX (US)

Correspondence Address: FISH & RICHARDSON P.C., P.O. BOX 1022, MINNEAPOLIS, MN, 55440-1022, US

(21) Appl. No.: 11/355,126

(22) Filed: Feb. 15, 2006

Related U.S. Application Data
(63) Continuation-in-part of application No. 11/091,127, filed on Mar. 28, 2005.

(51) Int. Cl. A61M 29/00

(52) U.S. Cl. 606/192

(57) ABSTRACT

A device for controlling the weight of a body comprises a passive or expandable member. The expandable version of the member includes an expandable and a non-expandable portion to its exterior surface. The member is selectively expanded and/or contracted following implanted within the abdominal cavity to provide pressure to the abdominal cavity, thereby restricting food intake without physically invading the abdominal cavity. The expandable version of the member is expanded and/or contracted following implantation to vary the amount of pressure provided to the abdominal cavity. The member is preferably positioned superficial to the fascia, muscle, peritoneum and abdominal cavity of the abdominal region according to the method of the present invention to minimize the risks associated with traditional food intake restriction surgeries.

### CROSS-REFERENCE TO RELATED APPLICATION

This application is a continuation-in-part of, and therefore claims priority from, U.S. patent application Ser. No. 11/091,127, filed on Mar. 28, 2005, which is incorporated herein by reference for all purposes.

### TECHNICAL FIELD

This invention relates to weight control, and more particularly to a device and method for controlling body weight via indirect reduction of the volume of the abdominal cavity.

### BACKGROUND

Obesity is a chronic disease and constitutes a major health concern. In the United States alone obesity accounts for more than $100 billion in health care annually. Far from being a purely cosmetic issue, being obese or morbidly obese puts a person at an increased risk for developing and/or aggravating dozens of serious medical conditions. More than 30 obesity-related medical conditions are currently recognized. These include arthritis, several forms of cancer, carpal tunnel syndrome, cardiovascular disease, gallbladder disease, gout, hypertension, infertility, liver disease, low back pain, obstetric and gynecologic complications, sleep apnea, stroke, type-2 diabetes, and urinary stress incontinence.

Obesity is commonly measured by using the Body Mass Index (BMI). In terms of BMI, obesity is defined as having a BMI of 30 kg/m². Morbid obesity is defined as the condition of obesity coupled with one or more secondary debilitating factors, such as hypertension, cardiovascular disease and/or diabetes. A BMI of 40 kg/m² is generally recognized to constitute morbid obesity. Importantly, morbid obesity ranks second only to smoking as a preventable cause of death in the U.S.

While obesity is recognized to be simply an imbalance between caloric intake and caloric burn rate, the factors producing obesity are varied and complex. Genetic, biological and even psychological influences can influence the condition. As a result, obesity is a disease that eludes simple treatment or attempts to shed weight.

Weight loss is generally recommended for persons with obesity or morbid obesity. The loss of excess weight can improve the health of a person by lowering risks from obesity-related medical conditions. Methods of weight loss include dietary therapy, increased physical activity, behavior therapy, drug therapy, surgery or a combination of therapies.

Attempts at sustained weight loss via non-surgical means within the population of the obese are overwhelmingly unsuccessful. Moreover, it is estimated that this disease has a recurrence rate in greater than 90%. Consequently, long-term results of conservative treatments for obesity are generally unsuccessful, and can actually prove detrimental by producing further loss of self-esteem with the regaining of weight.

In contrast, surgery is a well-established method of long-term weight control for persons with obesity. Surgical procedures assist a person in losing weight by adjusting the way the body digests and/or absorbs calories. This is most often accomplished via surgically-implemented changes to the stomach and/or small intestine.

One general category of obesity surgery targets the relative absorption of food. This type of procedure seeks to shorten the length of, or otherwise modify, the small intestine to limit the amount of foods that is ultimately absorbed by the body (malabsorption). Common examples of malabsorption procedures include: gastric bypass (e.g., Roux-en-Y gastric bypass); biliopancreatic diversion; and intestinal bypass.

Other surgical methods address obesity via restriction of food intake. This type of surgical procedure seeks to alter the size (volume) of the stomach, therefore limiting the amount of food it can hold. The result is a premature feeling of satiety and a reduced intake of calories. Common examples of procedures producing food intake restriction include: vertical banded gastroplasty; gastric banding; and laparoscopic gastric banding.

Through malabsorption, food intake restriction, or some combination of both, weight is reduced since less food either enters the stomach and/or less food remains in the small intestine long enough to be digested and absorbed.

As with any surgical procedure, there are risks associated with obesity surgical procedures. Additionally, each procedure has an associated success rate that, to a certain extent, is dependent upon whether a person is willing to make certain lifestyle changes in association therewith. As a general rule, procedures that invasively alter the size or volume of the stomach carry with them increased risks, such as infections, leaking of stomach juices into the abdomen, injury to the spleen, band slippage, erosion of the band, breakdown of the staple line, and stomach pouch stretching from overeating. Such risks are due not only to the physical stapling, banding or other direct manipulation of the stomach, but also in part to the fact that the surgeon has to invade the skin, fat, fascia, muscle and peritoneum of the abdominal region to make physical contact with the abdominal cavity to conduct such procedures.

In a previously unrelated area, tissue expanders have been employed in the context of cosmetic and reconstructive surgery where the need for additional tissue is present. Tissue expanders are implantable devices capable of expansion over time. They take advantage of the fact that tissue under prolonged physical stress will produce additional tissue. Such devices are used to dissect tissue, create cavities or pockets, or separate layers of soft tissue. In use for tissue dissection, for example, a surgeon makes a remote incision into the body and inserts a hollow tissue expander into the incision to a point where a space or cavity or pocket is desired. Fluid is then forced into the expander to cause it to expand and separate two layers of tissue to form the desired space or cavity or pocket. The dissection takes place along the edges of the incision and peripherally outward from the tissue expander.

Although there have been many improvements in tissue expanders since their inception, use of tissue expanders to date has been limited to dissecting tissue, creating cavities or pockets, or separating layers of soft tissue, and the like.

There remains a need for a device and method of addressing obesity that includes only minimally-invasive surgical procedures (thus avoiding many of the associated risks of surgical procedures that invasively alter the size of the stomach), but which produce success rates in terms of weight reduction and sustainability of same comparable to current, fully-invasive surgical procedures. Ideally, such a device and related method would reduce the volume of the abdominal cavity with a minimal amount of physical invasion of the abdominal cavity.

### SUMMARY

The present invention comprises a device and method for controlling body weight via indirect reduction of the volume of the abdominal cavity utilizing a specialized tissue expander. One embodiment of the present invention includes a device for producing intra-abdominal pressure from a point superficial to the fascia of the abdominal cavity to decrease the volume of the abdomen without physically invading the abdominal cavity. In an embodiment of the method of the present invention, an incision is made in the abdominal region of the body. Next a restriction device is placed into the incision to a point superficial to the fascia of the abdominal region. The presence of the restriction device creates intra-abdominal pressure, effectively reducing the volume of the abdominal cavity.

One embodiment of the restriction device is passive in nature. Upon placement, the passive restriction device exerts intra-abdominal pressure, reducing the volume of the abdominal cavity and thus reducing the amount of food required to experience a sense of “fullness”.

Another embodiment of the restriction device is capable of being expanded subsequent to implantation. Following placement, the expandable version of the restriction device is expanded to produce additional intra-abdominal pressure, effectively reducing to a greater extent the volume of the abdominal cavity.

Using either the passive or expandable version of the restriction device, placement and use of a restriction device increases the intra-abdominal pressure, effectively reducing the volume of food needed by the person to feel “full.” The ingestion of less food by the person will also necessarily advantageously affect relative food absorption in the lower intestine. Both contribute to weight loss. Importantly, such intra-abdominal pressure is created by either version of the restriction device without physical invasion of the abdominal cavity.

An embodiment of the expandable version of restriction device of the present invention is an expandable hollow member including means for expansion. Optimally, the hollow member includes both a rigid and a flexible region along its exterior surface. This feature, when the hollow member is oriented properly and expanded Via the means for expansion, will direct the pressure created by the expansion of the hollow member towards the abdominal cavity. Such pressure will effectively reduce the volume of the abdominal cavity, causing the person to require less food to achieve a sense of satiety.

Once in place using the present invention method, the level of expansion of the expandable version of the restriction device (hollow member) of the present invention can be selectively adjusted via the means for expansion without the need for additional surgery. If, for example, weight loss is occurring too rapidly for the person, the expansion can be reduced. The effect of the reduction of expansion will be a reduction of intra-abdominal pressure created by same, allowing the person to ingest a higher volume of food. If, on the other hand, weight loss is not progressing or has reached a plateau, expansion of the device can be increased, thereby increasing the intra-abdominal pressure created and further reducing the volume of food needed by the person to feel “full.”

The details of one or more embodiments of the invention are set forth in the accompanying drawings. and the description below. Other features, objects, and advantages of the invention will be apparent from the description and drawings, and from the claims.

### DESCRIPTION OF DRAWINGS

FIG. 1 is a cross section of an abdominal cavity of a human illustrating normal anatomy of same;

FIG. 2 is a cross section of an abdominal cavity of a human showing normal anatomy and including an embodiment of the system of the present invention (non-expanded state of expandable version or passive version);

FIG. 3 is a flow chart outlining the steps of an embodiment of the method of the present invention; and

FIG. 4 is a cross section of an abdominal cavity of a human showing normal anatomy and including an embodiment of the system of the present invention with the expanded version of the restriction device expanded.

Like reference symbols in the various drawings indicate like elements.

### DETAILED DESCRIPTION

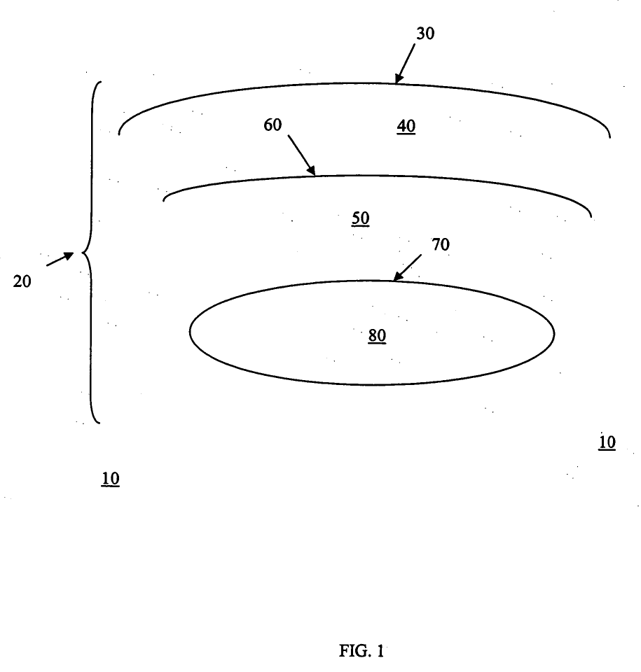

The present invention comprises a device and method for controlling body weight via indirect reduction of the volume of the abdominal cavity utilizing a specialized tissue expander. FIG. 1, a cross section of an abdominal cavity of a human exhibiting normal anatomy, illustrates a body 10, said body including an abdominal region 20 having skin 30, fat 40, muscle 50, fascia 60, a peritoneum 70 and an abdominal cavity 80. The abdominal cavity 80 comprises the stomach (not shown) which receives and processes food and other nourishment for the body 10, passing same to the intestines (not shown) of the body 10.

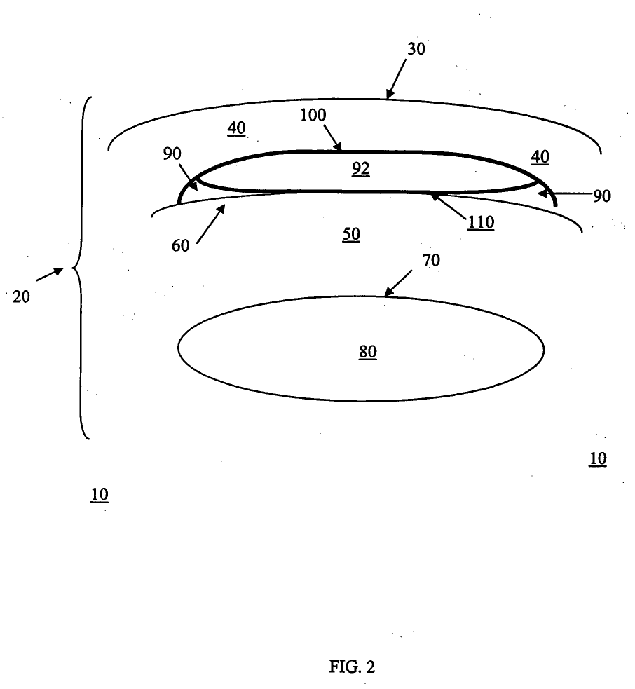

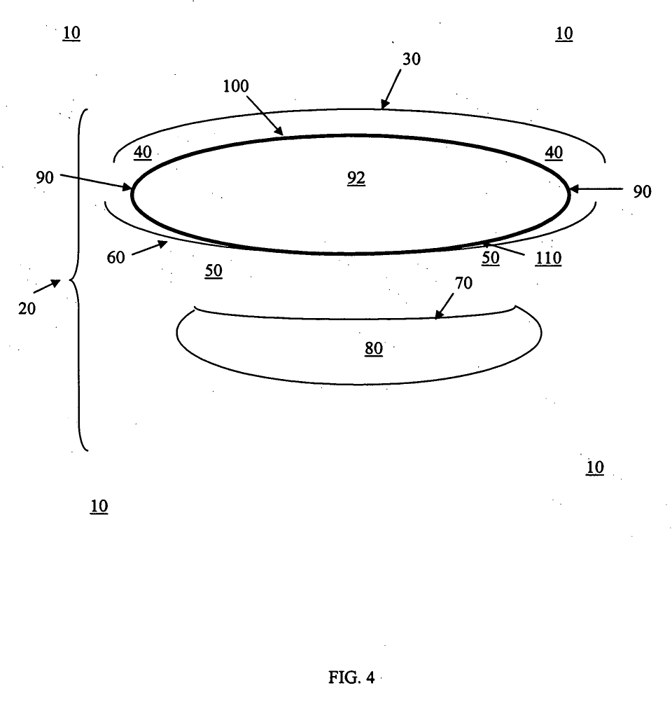

FIG. 2 illustrates a cross sectional view of the abdominal region 20 of the body 10 including placement of an embodiment of an implantable food restriction device 90 of the present invention (the expandable version in non-expanded mode). This version of the implantable food restriction device 90 is preferably comprised of a hollow member 92 and means for expansion and contraction (not shown) of the hollow member 92. The hollow member 92 preferably includes a relatively non-expandable portion 100 and a relatively expandable portion 110. When placed in the abdominal region 20 of the body 10, the relatively expandable portion 110 is oriented towards the abdominal cavity 80, thereby positioning the relatively non-expandable portion 100 of the hollow member 92 away from the abdominal cavity 80 (i.e., towards the fat 40). Once placed within the abdominal region 20 and expanded (see FIG. 4), the hollow member 92 will apply the force of pressure created from the expansion towards the fascia 60, the muscle 50, the peritoneum 70 and, ultimately, the abdominal cavity 80, thereby reducing the relative volume of the abdominal cavity 80. Although the hollow member 92 of the implantable food restriction device 90 of this embodiment is described as having a relatively expandable portion 110 and a relatively non-expandable portion 100, it is noted that the implantable food restriction device 90 can utilize a hollow member 92 lacking the relatively expandable portion 110 and/or the relatively non-expandable portion 100. Any expansion of the implantable food restriction device 90 will create intra-abdominal pressure that will reduce the relative volume of the abdominal cavity 80. Therefore, any suitable design of the implantable food restriction device 90 is contemplated by this invention.

FIG. 2 also illustrates the placement location of the passive version of the implantable food restriction device 90. The implantable food restriction device 90 can effectuate weight loss as a passive member, without the need for any expansion when implanted. The mere presence of a passive member, such as the implantable food restriction device 90, will create a force that is applied towards the fascia 60, the muscle 50, the peritoneum 70 and, ultimately, the abdominal cavity 80. Like with the expandable version of the implantable food restriction device 90, the passive version will create intra-abdominal pressure that will reduce the relative volume of the abdominal cavity 80, thereby reducing the relative food intake associated with an individual feeling “full”.

The passive version of the implantable food restriction device 90 may be constructed of any appropriate materials, such as medical grade polypropylene mesh or the like. Additionally, the relative size and dimensions of the passive version of the implantable food restriction device 90 may be varied to vary the intra-abdominal pressure created from the use of same. Although a polypropylene mesh is described above, it is noted that the passive implantable food restriction device 90 may be constructed of any appropriate material or materials, including medical grade plastics, nylon, polyester, and even metals (stainless steel).

One embodiment of the expandable version of the implantable food restriction device 90 includes means for expansion and contraction (not shown). Such means for expansion and contraction can be any suitable means for providing and extracting a liquid or gas from the hollow member in a manner that does not require additional surgical procedures. For example, one such means is a tube that is relatively hollow and flexible and which protrudes from, or is contained within, the body when attached to the hollow member 92 of the implantable food restriction device 90. The tube is utilized to provide and extract liquids or gases from the hollow member 92. If a liquid or gas is provided to the hollow member 92, it expands and creates the intra-abdominal pressure that reduces the relative volume of the abdominal cavity 80.

In the expandable version of the implantable food restriction device 90, the intra-abdominal pressure created by the hollow member 92 of the implantable food restriction device 90 can be controlled over time by providing and/or extracting a liquid or gas from the hollow member 92 in response to weigh loss progress of the body I0. Although a tube means has been described herein, it is noted that any suitable means for providing/extracting a liquid or gas from the hollow member 92 of the implantable food restriction device 90 may be employed with the present invention.

Either version of the food restriction device 90 of the present invention can be constructed of any suitable material(s), and is preferably constructed of a durable, relatively flexible material or materials capable of being safe use for prolonged periods of time within the human body, such as surgical grade plastics, polymers and the like. Additionally, both the static and expandable version of the food restriction device 90 may be sized and shaped in any suitable combination to produce the desired level of intra-abdominal pressure necessary to achieve desired weight loss by the body 10. It is noted that varying sizes, shapes and combinations thereof of the food restriction device 90 may be employed as suggested by the individual needs of the body 10 in question to optimize the results achieved via use of the present invention.

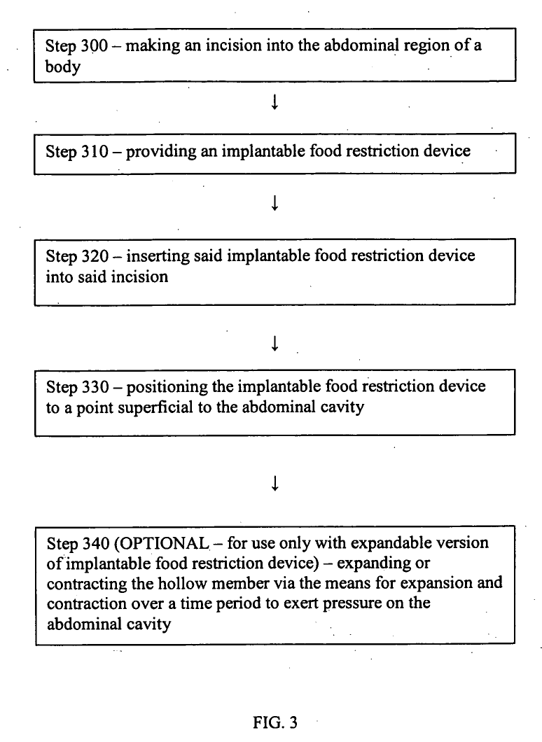

As illustrated in FIG. 3, one embodiment of the present invention surgical method for controlling weight of a body, said body including an abdominal region having skin, fat, muscle, fascia, a peritoneum and an abdominal cavity, comprises five primary steps. In Step 300 of the method, an incision is made into the abdominal region of a body. The incision can be made utilizing any traditional means for same, including via scalpel, laser, or other suitable cutting device. In Step 310, an implantable food restriction device is provided, said implantable food restriction device having either a passive form (e.g., mesh) or an expandable form (e.g., a hollow member and associated means for expansion and contraction). The implantable food restriction device is inserted in Step 320 into the incision created in step 300. The food restriction device of the present invention may be inserted via any suitable method for same, including, without limitation, via endoscope or open method technique. In Step 330, the implantable food restriction device is positioned to a point superficial to the abdominal cavity. In optional Step 340 (for use only with the expandable version of the food restriction device 90), the hollow member of the expandable version of the implantable food restriction device is selectively expanded or contracted via the means for expansion and contraction over a time period to exert pressure on the abdominal cavity.

The result of exerting pressure on the abdominal cavity is to control the relative volume of same. Increased pressure on the abdominal cavity will reduce its relative volume, causing the body (e.g., person) to require less food to achieve a sense of satiety. The ingestion of less food by the body will also necessarily advantageously affect relative food absorption in the lower intestine. Importantly, the hollow member of the expandable version of the implantable food restriction device 90 can be expanded or contracted without the need for additional surgery, allowing for highly flexible control over weight gain for the body. If, for example, weight loss is occurring too rapidly for the body, the expansion can be reduced. The effect of the reduction of expansion will be a reduction of intra-abdominal pressure created by same, allowing the person to ingest a higher volume of food (and also adjusting the relative food absorption rate in the lower intestine). The overall result will be a slow down in weight loss experienced by the body. If, on the other hand, weight loss is not progressing at a satisfactory rate (e.g., weight loss has reached a plateau), expansion of the implantable food restriction device 90 can be increased, thereby increasing the intra-abdominal pressure created, reducing the volume of food needed by the person to feel “fill,” and advantageously affecting the relative food absorption rate in the lower intestine.

FIG. 4 is an illustration of layers the abdominal region 20 of a body 10 including placement of an embodiment of the expandable version of the food restriction device 90 of the present invention (in expanded mode). As the hollow member 92 of the food restriction device 90 is expanded via the means for expansion and contraction (not shown), in a preferred embodiment the relatively expandable portion 110 of the hollow member 92 expands and directs the intra-abdominal pressure created by expansion of the food restriction device 90 towards the muscle 50 and the fascia 60 of the abdominal cavity 20, which in turn apply pressure to the peritoneum 70 and, ultimately, the abdominal cavity 80, the overall effect of which is to reduce the relative volume of the abdominal cavity 80 without the need for physical invasion of the abdominal cavity 80. With the relative volume of the abdominal cavity 80 reduced, the body 10 will require less food to achieve a sense of satiety. Additionally, the ingestion of less food by the body 10 will also necessarily advantageously affect relative food absorption in the lower intestine. The combined effect of the foregoing will be a reduction in weight of the body 10.

In use, the food restriction device 90 is positioned to a point superficial to the abdominal cavity 80. Specifically, in a preferred embodiment, the food restriction device 90 is positioned to a point superficial to the fascia 60 of the abdominal region 20. This positioning of the food restriction device 90 is considered an optimal balance of the desired transfer of intra-abdominal pressure to the abdominal cavity 80 with the desire to have the least physical invasion of the abdominal region 20. Although optimal positioning of the food restriction device 90 is described as being superficial to the fascia 60 of the abdominal region 20, it is noted that the invention contemplates positioning of the food restriction device 90 at any point within the abdominal region 20 that reduces the relative volume of the abdominal cavity 80 via the production of intra-abdominal pressure without physical invasion of the abdominal cavity 80, including, without limitation, points deep to the skin and the fat of the abdominal region, but (a) superficial to the fascia, the muscle and the peritoneum of the abdominal region; or (b) underneath the fascia; or (c) within the peritoneal cavity.

As can be appreciated, the change in relative volume of the abdominal cavity 80 created as a result of the food restriction device 90 of the present invention is directly related to the relative volume of the food restriction device 90 employed. Therefore, if a passive version of the food restriction device 90 is utilized, the relative shape and size can be varied to effectuate the desired change in volume of the abdominal cavity 80. If an expandable version of the food restriction device 90 is utilized, again the relative shape, construction (e.g., relative position of the relatively expandable portion 110 and/or the relatively non-expandable portion 100), and degree of expansion can be varied to effectuate the desired change in volume of the abdominal cavity 80. For example, with respect to the expandable version of the food restriction device 90, if additional weight loss by the body 10 is desired, the relative volume of the hollow member 92 of the food restriction device 90 can be increased via the means for expansion and contraction. Conversely, if it is desired that the body 10 experience less weight loss (or have the rate of weight reduction slowed), the relative volume of the hollow member 92 of the food restriction device 90 can be reduced via the means for contraction and expansion.

One significant advantage of the device and method of the present invention is that it does not invade the abdominal cavity 80. The lack of physical invasion of the abdominal cavity 80 eliminates leaking of stomach juices into the abdomen, injury to internal organs (e.g., spleen) and further avoids complications associated with the use of devices within the abdominal cavity 20 such as staples, bands and the like, thus leading to quicker recovery times, reduced hospital stays and increased patient satisfaction.

A number of embodiments of the invention have been described. Nevertheless, it will be understood that various modifications may be made without departing from the spirit and scope of the invention. For example, the device could be shaped or otherwise modified (e.g., in size) so as to more completely focus the pressure created via expansion of same. Accordingly, other embodiments are within the scope of the following claims.

What is claimed is:

1. An implantable device for use with a surgical method for controlling weight of a body having an abdominal cavity, said implantable device comprising:
a member;
said member shaped and sized to exert and direct intra-abdominal pressure within the body.

2. The implantable device of claim 1, wherein the member exerts and directs intra-abdominal pressure without invasion of the abdominal cavity of the body.

3. A surgical method for controlling weight of a body, said body including an abdominal region having skin, fat, muscle, fascia, a peritoneum and an abdominal cavity, the method comprising the steps of:
making an incision into the abdominal region of the body;
providing an implantable food restriction device;
inserting the implantable food restriction device into said incision; and
positioning the implantable food restriction device to a point superficial to the abdominal cavity to exert and direct pressure to the abdominal cavity.

4. The surgical method of claim 3, wherein the step of positioning the implantable food restriction device includes locating the implantable food restriction device at any point superficial to the abdominal region.

5. The surgical method of claim 3, wherein the step of positioning the implantable food restriction device includes locating the implantable food restriction device at a point deep to the skin and the fat of the abdominal region, but superficial to the fascia, the muscle and the peritoneum of the abdominal region.

6. The surgical method of claim 3, wherein the step of positioning the implantable food restriction device includes locating the implantable food restriction device at a point deep to the skin and the fat of the abdominal region, and underneath the fascia.

7. The surgical method of claim 3, wherein the step of positioning the implantable food restriction device includes locating the implantable food restriction device at a point deep to the skin and the fat of the abdominal region, and within the peritoneal cavity.

8. The surgical method of claim 3, wherein the step of positioning the implantable food restriction device further includes orienting the implantable food restriction device such that pressure created by implantable food restriction device is directed towards the abdominal cavity.

9. The surgical method of claim 3, further including the step of removing the implantable food restriction device.

10. The surgical method of claim 3, wherein endoscope or open technique is utilized to implant the food restriction device.

11. The surgical method of claim 6, wherein endoscope or open technique is utilized to remove the food restriction device.

12. The surgical method of claim 3, wherein the step of positioning the implantable food restriction device includes placing the implantable food restriction device at any point superficial to the abdominal cavity.
